(19) United States
(12) Patent Application Publication
(10) Pub. No.: US 2018/0103901 A1
(43) Pub. Date: Apr. 19, 2018

(54) Multifunction modular strap for a wearable device

(71) Applicant: CU Wellness, Inc., Los Altos Hills, CA (US)

(72) Inventors: Shripal Gandhi, Pune (IN); Keyur Patel, Mountain View, CA (US); Anal Patel, Mountain View, CA (US); Susanto Purnama, Mountain View, CA (US)

(21) Appl. No.: 15/833,877

(22) Filed: Dec. 6, 2017

Related U.S. Application Data
(63) Continuation-in-part of application No. 15/725,837, filed on Oct. 5, 2017.
(60) Provisional application No. 62/409,250, filed on Oct. 17, 2016.

(51) Int. Cl. A61B 5/00; A61B 5/024; A61B 5/11

(52) U.S. Cl. CPC A61B 5/681; A61B 5/0015; G06F 1/163; A61B 5/1112; A61B 5/02438

(57) ABSTRACT

The present disclosure relates to the field of electronic engineering. The present disclosure envisages a multifunction modular strap that integrates multiple health monitoring devices. The multifunction modular strap comprises a health sensing module, an activity tracking module, a signal conditioning unit, a processing unit, a notification module, and a communication module. The health sensing module has a plurality of health sensors configured to sense a plurality of health parameters associated with a user. The activity tracking module comprises a pedometer, a sleep detection module, and a gesture detection module. The signal conditioning unit co-operates with the health sensing module and the activity tracking module. The processing unit co-operates with the signal conditioning unit, the health sensing module and the activity tracking module. The notification module co-operates with the processing unit and notifies the user. The communication module receives at least one communication signal from the processor and enables bi-directional communication with communicatively coupled device.

### CROSS REFERENCE TO RELATED APPLICATIONS

This is a continuation in part of U.S. patent application Ser. No. 15/725,837, filed on Oct. 5, 2017 from which, as applicable, priority is claimed under 35 U.S.C. Section 120. The U.S. patent application Ser. No. 15/725,837 takes the priority from the U.S. Provisional Patent Application No. 62/409,250, filed on Oct. 17, 2016. The contents of each of the aforementioned patent applications are hereby incorporated by reference in their entirety.

### FIELD

The present disclosure relates to the field of electronics engineering. Particularly, the present disclosure relates to modular straps having sensors.

### BACKGROUND

Conventionally, people who monitor their health conditions, frequently visit health care centers in order to perform routine tests for measuring biometric parameters such as body temperature, UV Exposure relating to Vitamin-D deficiency, Skin Conditions, heart rate, blood pressure, ECG (Electrocardiography), photoplethysmogram (PPG), glucose level, and body fat analysis. Frequent trips to the health care centre are not only inconvenient for the people but are also costly and time consuming. Further, to avoid frequent trips to the health care centre, a caretaker is usually hired for performing such routine tests which also turn out to be expensive and in certain scenarios the caretaker may not be able to provide a quality service. Additionally, an area of concern is the requirement for the health care centers to provide a quick response to emergency cases such as falling of a patient, irregular changes in the heartbeat rate, change in ECG, and no movement condition of a patient under observation.

Furthermore, a user typically has to use multiple health monitoring devices such as a blood pressure monitoring device, an ECG machine, pulse oxymetry and a pedometer. Conventionally, since the multiple health monitoring devices used for measuring biometric parameters are not integrated, therefore completely distributed records are generated which are difficult to maintain and track. Further, the use of multiple health monitoring devices having different power handling capabilities results in high power dissipation in totality. In addition, the multiple electronic devices occupy a considerable amount of space which is not desired during travel.

Conventionally, various wearable devices such as wristwatches have been developed with an added functionality of measuring biometric parameters such as body temperature, heart rate, blood pressure, ECG (Electrocardiography), photoplethysmogram (PPG), glucose level, and body fat analysis associated with the user, separately. These dual function wearable devices are a lot more complex and expensive than the conventional wearable devices. Further, for example, the primary function of the smart watch is to display time and if the primary function of the smart wristwatch fails, then the smart wristwatch is either repaired or replaced which increases the cost.

Therefore, there is felt a need of a multifunction modular strap that can be retrofittedly attached to the conventional wearable devices and alleviates or eliminates the above-mentioned drawbacks.

### OBJECTS

Some of the objects of the present disclosure, which at least one embodiment herein satisfies, are as follows.

It is an object of the present disclosure to ameliorate one or more problems of the prior art or to at least provide a useful alternative.

An object of the present disclosure is to provide a multifunction modular strap that is compact.

Another object of the present disclosure is to provide a multifunction modular strap that requires less power.

Still another object of the present disclosure is to provide a multifunction modular strap that can be retrofitted on different wearable devices.

Yet another object of the present disclosure is to provide a multifunction modular strap that instantly transfers the measured health parameters to a concerned authority or health care centre.

Other objects and advantages of the present disclosure will be more apparent from the following description, which is not intended to limit the scope of the present disclosure.

### BRIEF DESCRIPTION OF ACCOMPANYING DRAWING

A multifunction modular strap for a wearable device of the present disclosure will now be described with the help of the accompanying drawings, in which:

FIG. 1 illustrates a block diagram of a multifunction modular strap.

FIG. 2 illustrates a schematic view of the multifunction modular strap of FIG. 1;

FIG. 3 illustrates schematic view of the multifunction modular strap of FIG. 1 worn on a wrist of a user;

FIG. 4 illustrates another schematic view of the multifunction modular strap of FIG. 1 worn on the wrist of the user; and

FIG. 5 illustrates a flow process for sensing, analyzing, comparing and transmitting information using the multifunction modular strap of FIG. 1.

### DETAILED DESCRIPTION

The present disclosure envisages a multifunction modular strap for a wearable device. A preferred embodiment of the multifunction a multifunction modular strap, of the present disclosure will now be described in detail with reference to the accompanying drawing. The preferred embodiment does not limit the scope and ambit of the disclosure. The description provided is purely by way of example and illustration.

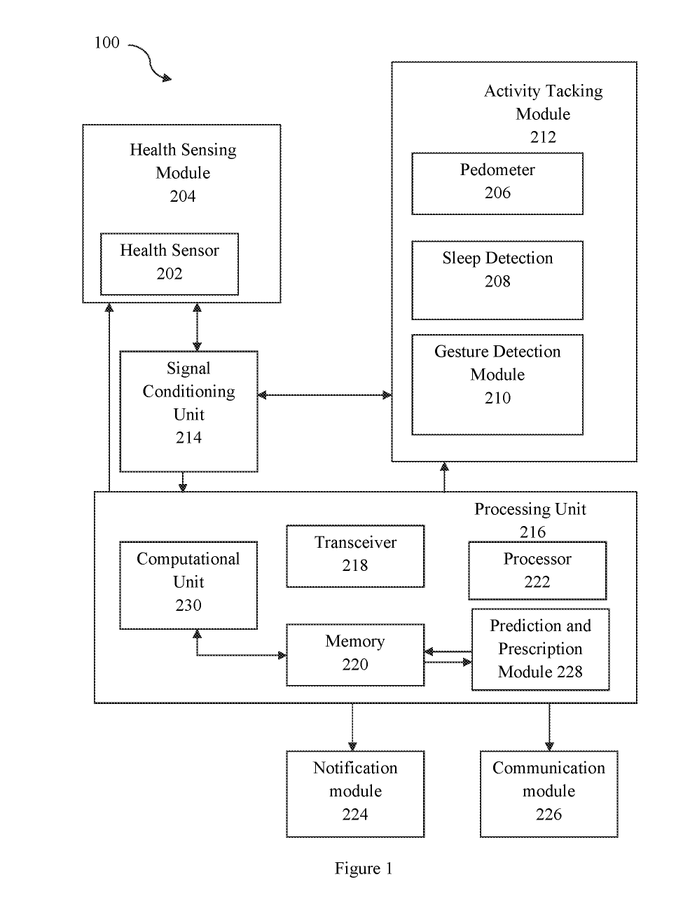

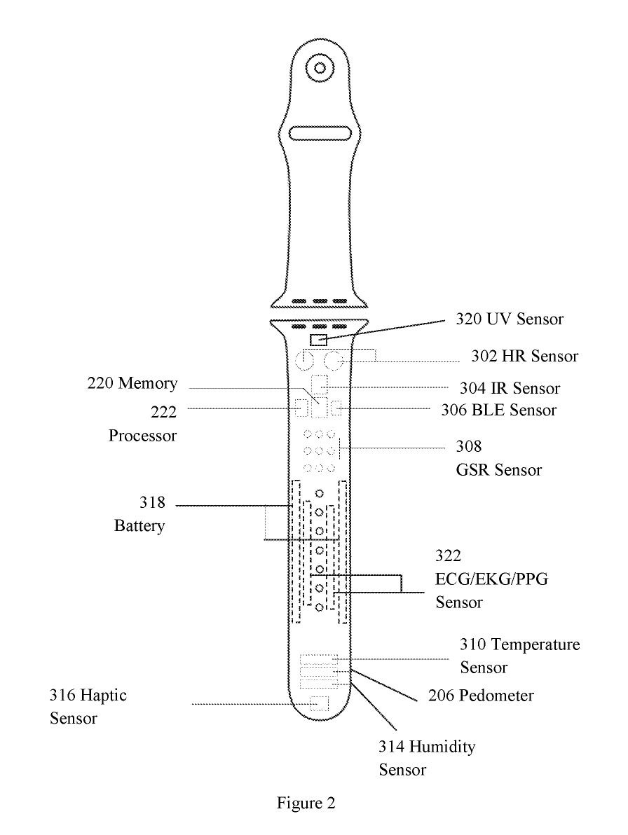

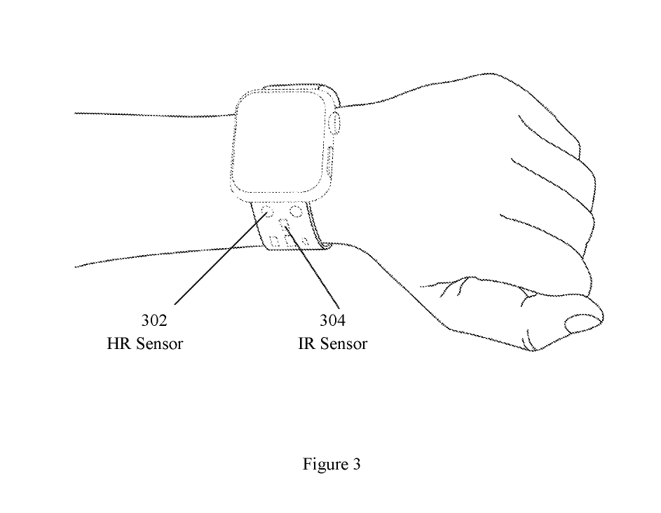

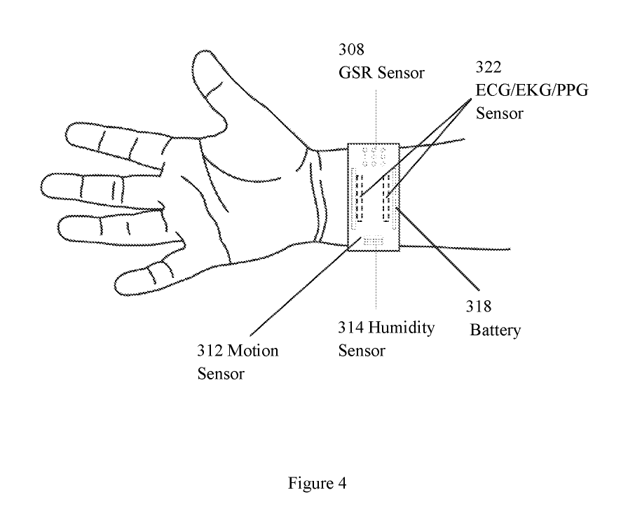

Referring to FIG. 1 through FIG. 4, a multifunction modular strap 100 for a wearable device comprises a health sensing module 204, an activity tracking module 212, a signal conditioning unit 214, a processing unit 216, a notification module 224, at least one battery 318, and a communication module 226. The at least one battery 318 is configured to provide power to the multifunction modular strap 100. In an embodiment, the at least one battery 318 is a flexible battery.

In an embodiment, the multifunction modular strap 100 facilitates wireless charging of the at least one battery 318. In another embodiment, the multifunction modular strap 100 facilitates wired charging of the at least one battery 318.

In an embodiment, the wearable device is a wristwatch.

The health sensing module 204 has a plurality of health sensors 202 configured to sense a plurality of health parameters associated with a user and generate a plurality of health signals. In an embodiment, the plurality of health sensors 202 is selected from the group consisting of calorie sensors, biometric sensors, heart-rate (HR) sensors 302, heart rate variability (HRV) sensors, blood oxygen sensors, sweat sensors, temperature sensors 310, humidity sensors 314, Galvanic skin response (GSR) sensors, emotion tracking sensors, stress tracking sensors, blood pressure monitoring sensors, Electrocardiogram (ECG/EKG) and/or photoplethysmogram (PPG) sensors 322, glucose level sensors, UV sensors 320, and skin conductance sensors.

In an embodiment, the blood oxygen sensors include a SpO2 (peripheral capillary oxygen saturation) sensor and/or a VO2 max sensor. The SpO2 sensor is configured to estimate the amount of oxygen in the blood. Specifically, it is the percentage of oxygenated haemoglobin (haemoglobin containing oxygen) compared to the total amount of haemoglobin in the blood (oxygenated and non-oxygenated haemoglobin). The VO2 max sensor is configured to measure the maximum amount of oxygen that an individual can utilize during intense or maximal exercise.

In another embodiment, the skin conductance sensor is a galvanic skin response (GSR) sensor 308. The GSR sensor 308 is configured to measure the electrical conductance of the skin, which varies with skins moisture level. As the sweat glands are controlled by the sympathetic nervous system, so moments of strong emotion, change the electrical resistance of the skin. Skin conductance is used as an indication of psychological or physiological arousal.

In an embodiment, the glucose level sensors include a transdermal sensor or optical sensor/an infrared (IR) sensor.

In another embodiment, the humidity sensor is placed at a location of the multifunction modular strap 100 suited for sensing one or more of a trans-epidermal water loss or sweat of the user during wearing the multifunction modular strap 100.

The activity tracking module 212 comprises a pedometer 206, i.e. a motion sensor, a sleep detection module 208, and a gesture detection module 210. The pedometer 206 is configured to detect a physical motion and count steps travelled by users, and is further configured to generate at least one pedometer signal. The sleep detection module 208 is configured to generate sleep signals that convey information related to one or more physiological functions that indicate sleep stage of the user. The gesture detection module 210 is configured to detect gestures of the user and generate a plurality of gesture signals. In an embodiment, the gesture detection module 210 includes at least one camera (not shown in figures) and at least one proximity sensor (not shown in figures). In an exemplary embodiment, the gesture detection module 210 is configured to perform remote operations on at least one communicatively coupled device, such as mobiles, tablets, laptop, personal-digital-assistant (PDA), and the like. In another embodiment, the remote operations is to activate a camera of the at least one communicatively coupled device for image capturing, video recording, and the like. In yet another embodiment, the remote operation is to control music, video, opening a document, and the like on the at least one communicatively coupled device.

The signal conditioning unit 214 co-operates with the health sensing module 204 and the activity tracking module 212. The signal conditioning unit 214 is configured to receive the plurality of health signals, the at least one pedometer signal, the sleep signals, and the plurality of gesture signals. The signal conditioning unit 214 is further configured to generate a conditioned health data. Additionally, the signal conditioning unit is configured to generate conditioned activity data, wherein the conditioned activity data includes pedometer data, sleep data, and gesture data.

The processing unit 216 co-operates with the signal conditioning unit 214, the health sensing module 204 and the activity tracking module 212. The processing unit 216 comprises a transceiver 218, a memory 220, a processor 222, and a computational unit 230. The transceiver 218 is configured to receive the plurality of conditioned health data and the conditioned activity data associated with the user when the wearable device is worn by the user. The memory 220 co-operates with the transceiver 218 and is configured to receive and store the plurality of conditioned health data and the conditioned activity data. The memory 220 is further configured to store a pre-determined set of threshold ranges associated with the user. Further, the processor 222 co-operates with the memory 220 and is configured to analyze and compare the plurality of conditioned health data and the conditioned activity data with the pre-determined set of threshold ranges stored in the memory 220. The processor 222 is further configured to generate at least one information signal for the notification module 224 and the communication module 226. In an embodiment, the processing unit 216 is an application specific integrated circuit (ASIC), a field programmable grid array (FPGA), an arm processor, and the like. In an embodiment, the at least one information signal is selected from the group consisting of notification signal and communication signal. In another embodiment, the processor 222 is further configured to generate a first activation signal for controlling the operation of the health sensing module 204. In still another embodiment, the processor 222 is configured to generate a second activation signal for controlling the operation of the activity tracking module 212.

In an embodiment, the computational unit 230 co-operates with the memory 220 and the processor 222. The computational unit 230 is configured to receive the conditioned health data and the conditioned activity data from the memory 220, and is further configured to analyze the conditioned health and activity data for determining a set of conditioned data using pre-determined set of rules stored in the memory 220.

Furthermore, the computational unit 230 is configured to determine:
a. metabolism of the user based on the set of conditioned data and a pre-determined metabolism computation technique;
b. stress level of the user and based on the set of conditioned data and a pre-determined stress computation technique; and
c. exposure to vitamin D of the user using the set of conditioned data and a pre-determined Vitamin-D exposure computation technique.

The notification module 224 co-operates with the processing unit 216. The notification module 224 is configured to receive the at least one information signal and provide notifications to the user based on the at least one information signal. In an embodiment, the notification module 224 includes at least one multi-color light emitting diode for providing visual notifications and at least one haptic sensor 316 for providing tactile notifications, i.e. haptic feedback. In an embodiment, the at least one multi-color light emitting diode is configured to provide color based indications pertaining to the health condition of the user. In one embodiment, the notification module 224 is configured to provide notifications to the user for events related to the at least one communicatively coupled device, such as mobiles, tablets, and the like. In another embodiment, the events related to the at least one communicatively coupled device are messages, calls, emails, and the like. In one embodiment, the communicatively coupled device may be remotely located.

The communication module 226 co-operates with the processing unit 216. The communication module 226 is configured to receive the at least one communication signal. The communication module 226 is further configured to enable a bi-directional communication with the at least one communicatively coupled device based on the at least one communication signal. In one embodiment, the communication module 226 includes a near field communication module (NFC) and a far field communication module. In an embodiment, the communication module includes an infrared (IR) sensor 304 and a Bluetooth lower energy (BLE) sensor 306.

In an embodiment, the processor 222 is configured to analyze and compare the health data and sleep data with the predetermined set of threshold ranges. If at least one of the health data, and the sleep data is above or below the predetermined set of threshold ranges, then the processing unit 216 sends the at least one notification signal and the at least one communication signal to the notification module 224 and the communication module 226 respectively. The notification module 224 is configured to notify the user by means of the multi-color light emitting diode for visual notification and the motor for tactile notification. The communication module 226 instantly transfers the health parameters associated with the user to the communicatively coupled device that may be remotely located. In an exemplary embodiment, the communicatively coupled device may be remotely located or at the user's health care center.

In an embodiment, the multifunction modular strap 100 includes a prediction and prescription module 228. The prediction and prescription module 228 is configured to predict sleep pattern, indigestion, and the progression of preventable chronic diseases which includes obesity, diabetes, hypertension, cardiovascular disease, and the like, based on at least one historical health data and at least one historical activity data associated with the user. In an embodiment, the prediction and prescription module 228 is configured to learn the patterns of the user based on the historical health data, historical activity data, and present health and activity data, and is further configured to generate predict health related information based on the learned patterns. In another embodiment, the prediction and prescription module 228 is configured to employ rule based techniques (formula based techniques) using historical health and activity data for predict health related information.

Further, the prediction and prescription module 228 is configured to provide health related recommendations to the user based on the predicted health related information.

In an embodiment, the aforementioned the health sensing module 204, the activity tracking module 212, the signal conditioning unit 214, the processing unit 216, the notification module 224, and the communication module 226 are fabricated as a flexible Printed Circuit Board Assembly (PCBA).

In another embodiment, the processor 222 is configured to analyze and compare the gesture data with the predetermined ranges stored in the memory 220. If the gesture data is within the predetermined set of range, then the processing unit 216 generates:
a first activation signal and activate the health sensing module 204; and
a second activation signal and activate the activity tracking module 212.

In yet another embodiment, the multifunction modular strap 100 is also configured to perform financial transactions.

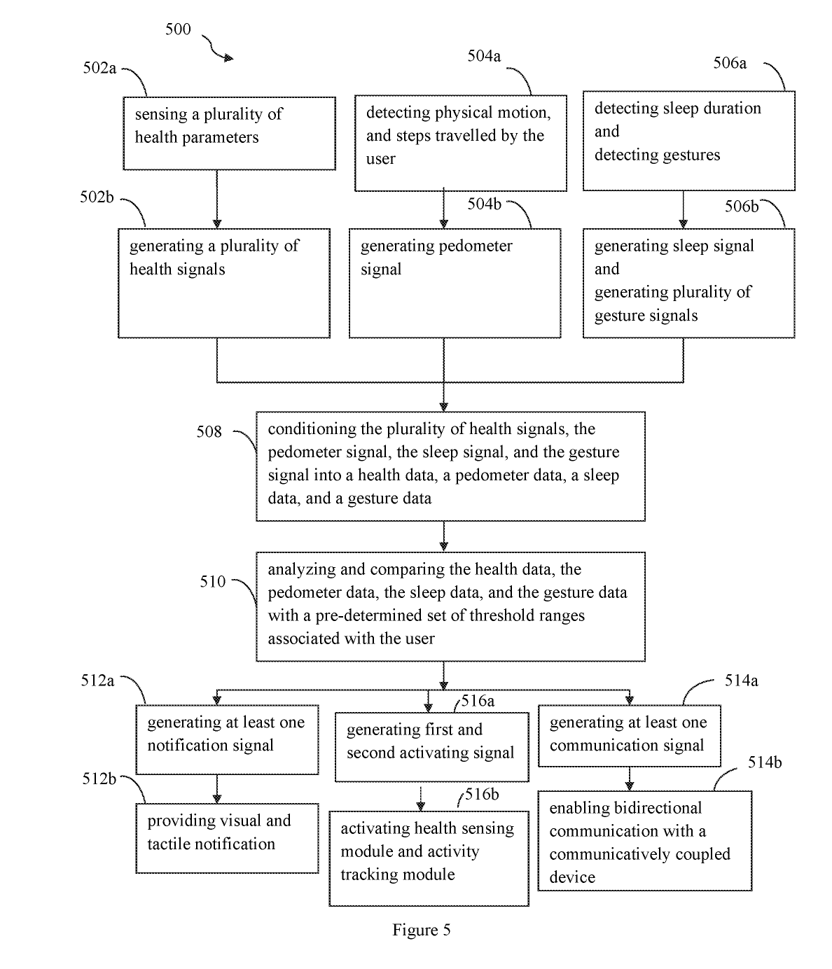

FIG. 5 illustrates a process 500 for sensing, analyzing, comparing and transmitting information using the multifunction modular strap 100.

Block 502a: sensing, by the plurality of health sensors 202, the plurality of health parameters associated with a user;

Block 502b: generating, by the plurality of health sensors 202, the plurality of health signals;

Block 504a: detecting, by the pedometer 206, physical motion and steps travelled by the user;

Block 504b: generating, by the pedometer 206, a pedometer signal;

Block 506a: detecting, by the sleep detection module 208 and the gesture detection module 210, sleep duration and gesture of the user;

Block 506b: generating, by the sleep detection module 208 and the gesture detection module 210, sleep signal and gestures signal;

Block 508: conditioning, by the signal conditioning unit 214, the plurality of health signals, the at least one pedometer signal, the sleep signals, and the plurality of gesture signal and generating health data, pedometer data, sleep data, and gesture data;

Block 510: analyzing and comparing, by the processor 222, the health data, the pedometer data, the sleep data, and the gesture data with the pre-determined set of threshold ranges;

Block 512a: generating, by the processor 222, at least one notification signal;

Block 512b: providing, by the notification module 224, visual and tactile notification;

Block 514a: generating, by the processor 222, at least one communication signal;

Block 514b: enabling, by the communication module 226, bidirectional communication with a communicatively coupled device;

Block 516a: generating, by the processor 222, first activation signal and second activation signal;

Block 516b: activating, by the processor 222, the health sensing module 204 and the activity tracking module 212.

### Technical Advances and Economical Significance

The present disclosure described herein above has several technical advantages including, but not limited to, the realization of a multifunction modular strap for a wearable device that:
requires less power;
cost effective;
integrates multiple health monitoring devices; and
instantly transfers the health parameters to the health care provider.

The disclosure has been described with reference to the accompanying embodiments which do not limit the scope and ambit of the disclosure. The description provided is purely by way of example and illustration.

The embodiments herein and the various features and advantageous details thereof are explained with reference to the non-limiting embodiments in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The foregoing description of the specific embodiments so fully revealed the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

The use of the expression “at least” or “at least one” suggests the use of one or more elements or ingredients or quantities, as the use may be in the embodiment of the disclosure to achieve one or more of the desired objects or results.

Any discussion of documents, acts, materials, devices, articles or the like that has been included in this specification is solely for the purpose of providing a context for the disclosure. It is not to be taken as an admission that any or all of these matters form a part of the prior art base or were common general knowledge in the field relevant to the disclosure as it existed anywhere before the priority date of this application.

The numerical values mentioned for the various physical parameters, dimensions or quantities are only approximations and it is envisaged that the values higher/lower than the numerical values assigned to the parameters, dimensions or quantities fall within the scope of the disclosure, unless there is a statement in the specification specific to the contrary.

While considerable emphasis has been placed herein on the components and component parts of the preferred embodiments, it will be appreciated that many embodiments can be made and that many changes can be made in the preferred embodiments without departing from the principles of the disclosure. These and other changes in the preferred embodiment as well as other embodiments of the disclosure will be apparent to those skilled in the art from the disclosure herein, whereby it is to be distinctly understood that the foregoing descriptive matter is to be interpreted merely as illustrative of the disclosure and not as a limitation.

1. A multifunction modular strap for a wearable device, said multifunction modular strap comprising:
a health sensing module configured to sense a plurality of health parameters, associated with a user, and further configured to generate a plurality of health signals;
an activity tracking module configured to sense a plurality of activity parameters, associated with said user, and further configured to generate a plurality of activity signals;
a signal conditioning unit co-operating with said health sensing module and said activity tracking module and configured to generate a plurality of conditioned health data and a plurality of conditioned activity data based on said plurality of health and activity signals respectively;
a processing unit co-operating with said signal conditioning unit to receive said conditioned health data and conditioned activity data, and configured to generate at least one information signal based on said conditioned health data and conditioned activity data; and
a notification module co-operating with said processing unit, and configured to receive said at least one information signal and provide notifications to said user.

2. The multifunction modular strap as claimed in claim 1, wherein said activity tracking module includes:
a pedometer configured to detect a physical motion and count steps travelled by said user and further configured to generate at least one pedometer signal;
a sleep detection module configured to generate sleep signals that convey information related to one or more physiological functions that indicate sleep stage of said user; and
a gesture detection module configured to detect gestures of said user and generate a plurality of gesture signals.

3. The multifunction modular strap as claimed in claim 1, wherein said plurality of activity signals include the at least one pedometer signal, the sleep signal, and the plurality of gesture signals.

4. The multifunction modular strap as claimed in claim 2, wherein said gesture detection module includes at least one camera, and at least one proximity sensor.

5. The multifunction modular strap as claimed in claim 2, wherein said signal conditioning unit is configured to receive said plurality of health signals, said at least one pedometer signal, said sleep signals, and said plurality of gesture signals and generate a corresponding conditioned health data, and conditioned activity data.

6. The multifunction modular strap as claimed in claim 1, wherein said processing unit comprises:
a transceiver configured to receive said plurality of conditioned health data and said plurality of conditioned activity data associated with said user;
a memory configured to cooperate with said transceiver and receive and store said plurality of conditioned health data and said plurality of conditioned activity data, and further configured to store a pre-determined set of threshold ranges associated with said user; and
a processor co-operating with said memory and configured to analyze and compare said plurality of conditioned health data and said plurality of conditioned activity data based on said pre-determined set of threshold ranges and further configured to generate said at least one information signal.

7. The multifunction modular strap as claimed in claim 6, wherein said at least one information signal is selected from the group consisting of notification signal and communication signal.

8. The multifunction modular strap as claimed in claim 6, wherein said processor is selected from the group consisting of an application specific integration circuit (ASIC), an FPGA (field programmable gate array), an embedded processor, an ARM processor, a PIC controller, and combination thereof.

9. The multifunction modular strap as claimed in claim 6, wherein said processor is further configured to generate a first activation signal for controlling the operation of said health sensing module.

10. The multifunction modular strap as claimed in claim 6, wherein said processor is further configured to generate a second activation signal for controlling the operation of said activity tracking module.

11. The multifunction modular strap as claimed in claim 1, wherein said multifunction modular strap further includes a communication module cooperating with said processing unit configured to receive said at least one information signal and further configured to enable a bidirectional communication with at least one communicatively coupled device based on said at least one information signal.

12. The multifunction modular strap as claimed in claim 11, wherein said communication module includes a near field communication module (NFC), and a far field communication module.

13. The multifunction modular strap as claimed in claim 11, wherein said communication module includes a Bluetooth lower energy (BLE) sensor and an Infrared (IR) sensor.

14. The multifunction modular strap as claimed in claim 1, wherein said plurality of health sensors is selected from the group consisting of calorie sensors, biometric sensors, heart-rate (HR) sensors, heart rate variability (HRV) sensors, blood oxygen sensors, sweat sensors, temperature sensors, humidity sensors, emotion tracking sensors, stress tracking sensors, blood pressure monitoring sensors, Electrocardiogram (ECG/EKG) and/or photoplethysmogram (PPG) sensors 322, glucose level sensors, UV sensors, and skin conductance sensors.

15. The multifunction modular strap as claimed in claim 1, wherein said notification module includes at least one multi-color light emitting diode for providing visual notification, and at least one motor for providing tactile notification.

16. The multifunction modular strap as claimed in claim 1, wherein said multifunction modular strap further includes at least one battery for providing power to said multifunction modular strap.

17. The multifunction modular strap as claimed in claim 16, wherein said multifunction modular strap is adapted to wirelessly charge said at least one battery.

18. The multifunction modular strap as claimed in claim 16, wherein said at least one battery is flexible.

19. The multifunction modular strap as claimed in claim 1, wherein said multifunction modular strap includes a prediction and prescription module which either employs a pattern based prediction technique or rule based prediction technique for predicting the health related information associated with the user, and is further configured to provide recommendations to the user based on said predicted health related information.

20. The multifunction modular strap as claimed in claim 1, wherein said processing unit includes a computational unit, said computational unit is configured to determine:
a. metabolism of the user based on said conditioned health data, conditioned activity data, and a pre-determined metabolism computation technique;
b. stress level of the user and based on said conditioned health data, conditioned activity data, and a pre-determined stress computation technique; and
c. exposure to vitamin D of the user using the conditioned health data and a pre-determined Vitamin-D exposure computation technique.
